(19) United States
(12) Patent Application Publication
(10) Pub. No.: US 2004/0146566 A1
(43) Pub. Date: Jul. 29, 2004

(54) Topical medication for burns and wounds

(76) Inventor: Robert E. Armstrong, Norwalk, CA (US)

Correspondence Address: Maria Erlinda C Sarno, PO Box 1023, Artesia, CA, 90702, US

(21) Appl. No.: 10/476,324

(22) PCT Filed: May 3, 2002
(86) PCT No.: PCT/US02/14027

(51) Int. Cl. A61K 35/20; A61K 35/64

(52) U.S. Cl. 424/535; 424/539; 424/195.18

(57) ABSTRACT

A medication for burns and wounds comprising beeswax in combination with butter and with, in most application, rosin, for providing immediate relief of pain and hastening the scabbing process. For medications with rosin, the formulation comprise 7-14% rosin, 10-50% beeswax and 50-70% butter preferably at a ratio of one part rosin, 2-4 parts beeswax, and 10-12 parts unsalted butter. All ingredients are natural and commercially available. The medication is stable at room temperature and is observed to increase its effectiveness or efficacy with storage.

### TECHNICAL FIELD

The present invention relates to a medication or composition for treating wounds generally caused by burns from flame, fire, boiling liquids and other chemical or electrical contact and by cuts and rashes from scratches, dry and chapped skin, sun burn, lacerations, psoriasis and the like. This medication prevents or substantially reduces scar formation after treatment. This composition can also be used as a carrier or in combination with other ingredients such as scents and perfumes, various additives of dermatological importance and applications, as well as drugs, and other chemical compounds including but not limited to antibiotics, analgesics, anaesthetics, vitamins and the like.

### BACKGROUND

Several antiburn creams are commercially available but they generally do not offer immediate relief from sensation of pain and usually do not prevent scar formation after healing. U.S. Pat. No. 6,099,866 (hereinafter '866) discloses a topical composition of fresh, unweathered, non-degraded beeswax, preferably from a single plant source, together with oil and water. Unlike the mentioned patent, the claimed invention comprises three major ingredients, beeswax, rosin and unsalted butter which are preferably all natural. These ingredients can all be purchased commercially. The claimed invention does not require a fresh beeswax but any commercial beeswax can be used. Further, '866 proposes to use the composition as a butter or margarine substitute which differs from the claimed invention. The claimed invention is not a substitute for butter or margarine but requires the addition of unsalted butter into the formulation. Likewise, the claimed invention does not combine the beeswax with oil and water and it also renders immediate relief of pain while '866 generally require about 20 minutes before its effect can be experienced by a burn patient. The claim invention does not require rubbing the medication into the affected area especially for burn patients but a gentle spreading over the surface of the affected area and allowing it to melt to the body temperature is recommended. Rubbing may worsen the pain and damage the affected area.

It is therefore an object of this invention to provide a medication or a composition for wounds caused by burn, cuts and rashes using a mixture of commercially available natural products.

It is also an object of this invention to provide a medication or a composition for wounds caused by burn, cuts and rashes which offers immediate relief of pain and curtails the bleeding of a wounded patient as is seen on burn patients.

It is a further object of this invention to provide a medication or a composition for wounds caused by burn, cuts and rashes which is stable for years at room temperature with increasing efficacy on long term storage. Efficacy means the ability of the medication to perform its claimed function or its effectiveness.

It is also a further object of this invention to provide a medication or a composition for wounds caused by burn, cuts and rashes that prevents or substantially reduce scar formation on the skin after healing.

It is also a further object of this invention to provide a medication for long term relief of psoriasis.

### DISCLOSURE OF INVENTION

The invention pertains to a wound healing composition or medication comprising beeswax, rosin and butter, the butter preferably grade AA and unsalted and the rosin, an athletic grade. The wound as used herein refer to those caused by burns from flame, fire, boiling liquids and other chemical or electrical contact and by cuts and rashes from scratches, dry and chapped skin, sun burn, lacerations, psoriasis and the like. The preferred ratio of the components or ingredients of this composition is one part rosin, 2-4 parts beeswax, and 10-12 parts unsalted butter. Increasing the concentration of beeswax increases its efficacy to heal the wound. However, with the increase in concentration of the beeswax, the composition hardens to a point where it would be difficult to apply over the affected areas. Doubling the concentration of beeswax would still result in an applicable medication. Rosin primarily causes the relief from pain. Doubling the concentration of rosin is recommended for some applications. For some applications that require exposure of the wound or rashes to air, for example, as a skin conditioner or for treatment of dry skin, the medication comprises beeswax and unsalted butter without the rosin because the latter is not recommended for dry skin. The rosin is believed to shield the wound from oxygen which would worsen the dry skin condition and would not be good for conditioning the skin. The ratio of beeswax to butter is preferably one part beeswax to four parts butter. A lower ratio of beeswax to butter reduces the healing power of the medication.

The method of preparing or compounding the composition comprise the steps of: preheating a vessel to approximately 120° C., the vessel preferably indirectly heated such as a double boiler, crock pot, and the like; melting a desired amount of butter or its equivalent, preferably unsalted, in the vessel, stirring the butter as it melts; cutting beeswax into chunks; adding the chunks of beeswax into the melted butter and stirring the mixture until the beeswax is fully melted and blended with the butter; adding a desired amount of rosin into the mixture of beeswax and butter; stirring the mixture until the rosin is melted and blended fully with the butter and beeswax; cooling the reaction vessel to approximately 100° C.; allowing the mixture to simmer at this temperature with stirring for approximately one hour; cooling further the resulting composition after simmering and pouring the composition into containers while the composition is still liquid; allowing the composition to cool to room temperature; and, closing the container for storage. The temperature of the vessel need not reach 150° C. before the butter is introduced into the vessel.

### DETAILED DESCRIPTION OF THE INVENTION

This invention relates to a composition or medication for treating wounds caused by burns, cuts and rashes which is stable at room temperature thereby allowing repeated usage without expiration. The composition usually comprises beeswax, butter and rosin. The healing power of the composition is mainly due to the beeswax. The beeswax can be of any type, so long as it is 100%. Commercially available beeswax is as equally suitable as any specially prepared beeswax. It does not matter whether the beeswax is obtained from fresh or aged combs, filtered or unfiltered, and no special treatment like freezing is necessary prior to the preparation of the composition or medication. The method of obtaining beeswax is known.

The butter used in preparing the composition is preferably grade AA and unsalted to prevent the stinging effect of salt on the open wound. Margarine and pasteurized cream may be used instead of butter but are not recommended. As in butter, these should preferably be unsalted. Examples of commercially available unsalted butter, are sweet unsalted butter sold by Challenge Dairy Products Inc., Dublin, Calif.; Land O'Lakes, Inc., Arden Hills, Minn. 55126; and, Alta Dena, 17637 E. Valley Blvd., City of Industry, Calif. 91747. Butter or its substitute is primarily added to soften and make the composition spreadable. However, because of the high fat and Vitamin A content of some if not all of these ingredients or component, they also contribute to the healing process of the wound.

Rosin, also known as colophony, is amber in color obtained as the residue in the distillation of turpentine coming preferably from pine trees, i.e. genus pinus trees. The rosin provides the immediate relief from pain which is subsequently aided and maintained by the beeswax. An athletic grade rosin is preferably used which is the rosin usually used in sports to prevent dampness or wetness of a pitcher's or catcher's mitt and are applied by players to prevent sweaty hands. The rosin is commercially available from Franklin Sports, Inc., Stoughton, Mass. 02072; BFB Enterprises, P.O. Box 28180, 393 Wehon Road, Panama City Beach, Fla. 32411; Mueller Sports Medicine, Inc., One Quench Drive, Prairie du Sac, Wis. 53578; and, in most sporting goods store.

The preferred ratio of these three ingredients relative to each other is one part rosin, 2-4 parts beeswax, and 10-12 parts unsalted butter. A medication can range from 7-10% rosin, 10-50% beeswax and 50-70% butter. The efficacy of the composition is dependent upon the beeswax concentration. Although an increase in the beeswax concentration will hasten the wound healing process, the amount of beeswax is limited by the ability to apply the composition or medication over the surface of the affected area. At the cited ratios above, the composition is soft and spreadable. Therefore, other ratios with at a concentration of approximately 10% to approximately 50% beeswax will still be effective if the physical state of the medication such as pourability, softness and spreadability is not an important factor to consider. For some conditions such as psoriasis, increasing the rosin concentration to as much as double its original concentration will hasten the scabbing process and its subsequent removal with the beeswax and butter healing the inner skin and restoring its original coloration. In example A, the rosin concentration is approximately 7%. An increase to 10% rosin while maintaining the amount of beeswax would be preferable for treatment of psoriasis. This medication or composition is very stable at room temperature and breakdown of the product due to bacterial contamination is prevented by the natural preservative property of beeswax owing to its antioxidant, antiacidification and antibacterial properties.

There are some application that may not require rosin such as those used as skin conditioner, cream for skin rashes caused by dry skin and eczema. For these medication, the ratio of beeswax to butter, one part beeswax to four parts butter, is preferable. Rosin is not recommended for this application because it shields the air from the skin surface.

There are normally no other ingredients added to the composition. However, one may add other ingredients to this composition such as scents and perfumes, various additives of dermatological importance and applications, as well as drugs, and other chemical compounds including but not limited to antibiotics, analgesics, anaesthetics, vitamins and the like. These preparations combine the benefit derived from the composition and the additive. For example, the medication combined with Aloe Vera, an additive with a dermatological application, will have the combined benefits of the medication and aloe vera, with the latter not necessarily contributing to the effects of the claimed medication. However, to maintain the efficacy of the resulting combination as a wound healer, the % concentration or the ratio of the beeswax to the other ingredients and additives must be maintained at the levels disclosed above.

A method of preparing the composition comprise the steps of: preheating a vessel to approximately 120° C., the vessel preferably indirectly heated such as a double boiler, crock pot, and the like; melting a desired amount of butter or its equivalent, preferably unsalted, in the vessel, stirring the butter as it melts; cutting beeswax into chunks; adding the chunks of beeswax into the melted butter and stirring the mixture until the beeswax is fully melted and blended with the butter; adding a desired amount of rosin into the mixture of beeswax and butter; stirring the mixture until the rosin is melted and blended fully with the butter and beeswax; cooling the reaction vessel with stirring to approximately 110° C.; holding the mixture at this temperature with stirring for approximately one hour; cooling further the resulting composition after holding and pouring the composition into containers while the composition is still liquid; allowing the composition to cool to room temperature; and, closing the container for storage. For preparations without rosin, the method above simply skips the addition of rosin.

The order of addition may vary without departing from the scope of this invention. However, to prevent the beeswax from sticking to the vessel, butter is melted first before the addition of the beeswax or rosin. Any method of blending the three ingredients may be employed. Example One shows a method of preparing the composition. All the examples herein are mainly provided for illustration and not as a limitation to the invention especially with regards to the relative concentration of the ingredients to each other and the process of formulating the composition such as the sequence of mixing the ingredients.

### EXAMPLE A

### A Process for 21.5 Oz. of Composition

Heat a vessel to approximately 120° C. and add 16 ounces (oz) of unsalted butter into the heated vessel. Continuous or occasional stirring of the butter while melting is preferred and for this example, this was done at 8-12 revolutions per minute at intervals of 2 minutes. The butter melts at about 50° C. Add 4 oz of beeswax which had been cut into chunks to the melted butter and stir the mixture while the beeswax melts at about 93° C. at the same speed as above. When the butter and beeswax are fully melted and blended, add approximately 1.5 Oz of rosin and stir the mixture until the rosin is fully melted and blended with the butter and beeswax. When the three ingredients or components are blended together, reduce the heat to about 110° C. and hold the mixture, i.e. simmer at this temperature for approximately one hour. Continuous or occasional stirring during this period is recommended. After approximately one hour, the resulting composition is slightly cooled and poured into containers. If a number of containers have to be filled, it is recommended to stir the composition while the containers are being filled to ensure that the rosin is evenly distributed. After filling, the containers containing the composition or medication are cooled to room temperature and sealed or covered for storage. Warmer temperature may cause the medication to liquefy while lower temperature may cause the medication to harden. However, these temperatures, although affecting the physical state of the medication, does not affect its efficacy.

If other ingredient/s is desired to be added to the composition, the above process can be easily modified by those in the art to accommodate the added ingredient. The point of addition will depend upon the characteristics or properties of the ingredient/s to be added such as solubility, miscibility, temperature stability, melting point if a solid, etc.

The composition of the invention has been found to be stable for a long period of time and more importantly, was observed to increase in efficacy after storage. Additionally, this composition also prevent or substantially reduce scar formation after the wound is healed. The medication is preferably packaged as an ointment or cream in a jar, vial or bottle; in a tubular packaging such as that used in toothpastes; or in the form of medicated patches.

This composition is generally applied to the affected area in a following manner: after a burn or cut, apply the composition as soon as possible by spreading the composition over the affected area. A flat object such as a butter knife, spatula, and the like, is preferably used to spread the medication over the entire affected area. A generous amount of the medication is preferably spread over the affected area. The composition melts to the body temperature and slowly penetrates the affected area without the need of rubbing. Rubbing may, especially for burn patient, worsen the affected area. After application, the affected area may be wrapped with a cloth, preferably soft and smooth such as those used for bed sheets, if desired. If the wound is due to contact with other hot materials that stick to the affected surface such as hot food, boiling liquids and the like, these materials are first washed off before applying the composition or medication. The medication can be applied to the affected area as much as 24 hours after the wound occurrence. The medication is usually applied two times a day and as needed on subsequent days. Unlike medications through prescription, the frequency of application is not a problem because all ingredients are natural and will not cause deleterious effects even if applied more frequently. For extensive burns or wounds, a body wrap may be required. For these, the amount of beeswax is reduced to increase its spreadability and rosin is increased for immediate relief of pain.

While the mode of application above may be used for all wounds, certain specific applications have also been applied on a case by case basis as recommended by patients such as the method used for the long term medication of psoriasis. In this case, the affected area is cleaned with soap and water and dried as thoroughly as possible. The medication is applied generously on the surface of the affected area with the finger or by the use of a flat object. The affected area is optionally wrapped with a cloth to protect a patient's clothing. In case of stains on one's clothing, use a stain remover if ordinary detergents won't work. Every evening, the affected area is cleaned and dried with a towel with rubbing to remove any detachable outer layer of the skin before reapplying the medication. This treatment is continued until the skin has returned to its original condition. On some instances, the skin may remain discolored or substantially improved with prolonged application of the medication. Once the skin has returned to its original condition, it is recommended to apply the medication once every 5 days to keep the skin from returning to it psoriatic condition.

While the embodiment of the present invention has been described, it should be understood that various changes, modifications and adaptations may be made therein without departing from the spirit of the invention and the scope of the appended claims. Those skilled in the art will recognize that other and further variations of the values presented herein are possible. The scope of the present invention should be determined by the teachings disclosed herein, the appended claims and their legal equivalents.

I claim:

1. A topical medication for burns and wound, comprising: a combination of beeswax, rosin and butter.

2. The medication of claim 1 wherein the beeswax is 100 percent pure.

3. The medication of claim 1 wherein the beeswax is commercially available pure beeswax.

4. The medication of claim 1 wherein the butter is substituted by margarine or pasteurized cream.

5. The medication of claim 1 wherein the butter is unsalted, grade AA and sweet.

6. The medication of claim 1 wherein the rosin is athletic grade rosin.

7. The medication of claim 1 wherein the rosin comes from genus pinus trees.

8. The medication of claim 1 wherein the ratio of rosin to beeswax to butter is one part rosin, 2-4 parts beeswax and 10-12 parts butter.

9. The medication of claim 1 wherein the beeswax is 10%-50% of the total weight of the medication.

10. The medication of claim 1 wherein the rosin is 7% to 14% of the total wight of the medication.

11. The medication of claim 1 further comprising additives selected from the group consisting of scents and perfumes, additives of dermatological importance and applications, drugs, antibiotics, analgesics, anaesthetics, vitamins and chemical compounds used in dermatology.

12. The medication of claim 1 wherein the medication is stable at room temperature and increases its efficacy on storage.

13. A topical medication for skin conditioning and rashes requiring exposure to air, comprising: a combination of pure commercial beeswax and unsalted butter.

14. The medication of claim 13 wherein the ratio of beeswax to butter is one part beeswax and four parts butter.

15. The medication of claim 13 wherein the beeswax is 10% to 50% of the total weight of the medication.

16. The medication of claim 13 wherein the butter is substituted by margarine or pasteurized cream.

17. The medication of claim 13 wherein the unsalted butter is commercially available, grade AA and sweet.

18. A topical medication for burns and wound, consisting essentially of: a combination of beeswax, rosin and butter.

19. A method for treating burns and wounds comprising the step of topically applying a composition of rosin, beeswax and butter to the affected area.

20. The method of claim 19 wherein the composition is 7-14% rosin, 10-50% beeswax and 50-70% butter.

21. A method of preparing a medication for burns and wound, comprising the steps of: preheating a vessel to approximately 120° C.; melting a desired amount of butter in the preheated vessel, stirring the butter as it melts; cutting beeswax into chunks; adding the chunks of beeswax into the melted butter and stirring the mixture until the beeswax is fully melted and blended with the butter; adding a desired amount of rosin, if needed, into the mixture of beeswax and butter; stirring the mixture until the rosin, if added, is melted and blended fully with the butter and beeswax; cooling the reaction vessel to approximately 110° C.; allowing the mixture to simmer at this temperature with stirring for approximately one hour; cooling further the resulting composition after simmering and pouring the composition into containers while the composition is still liquid; allowing the composition to cool to room temperature; and, closing the container for storage.
